(19) United States
(12) Patent Application Publication
(10) Pub. No.: US 2024/0390640 A1
(43) Pub. Date: Nov. 28, 2024

(54) BRAIDED STRUCTURE, TUBE STRUCTURE, TUBE STRUCTURE FOR CATHETER, METHOD FOR MANUFACTURING BRAIDED STRUCTURE

(71) Applicant: Japan Lifeline Co., Ltd., Tokyo (JP)

(72) Inventors: Takuya SASAKI, Tokyo (JP); Hiroki KAMIYAMA, Tokyo (JP)

(21) Appl. No.: 18/788,593

(22) Filed: Jul. 30, 2024

Related U.S. Application Data
(63) Continuation of application No. PCT/JP2023/000047, filed on Jan. 5, 2023.

(30) Foreign Application Priority Data
Mar. 7, 2022 (JP) 2022-034270

(51) Int. Cl. A61M 25/00

(52) U.S. Cl. CPC A61M 25/005; A61M 25/0012

(57) ABSTRACT

A catheter includes a shaft, a first wire wound in a spiral shape around an axis of the shaft, and a second wire wound around the shaft in a direction opposite to that of the first wire in a first portion, and wound around the shaft in a direction identical to that of the first wire in a second portion. Accordingly, it is possible to provide a braided structure and the like having flexibility that differs depending on the area in an axial direction.

### CROSS REFERENCE TO RELATED APPLICATIONS

This application is a continuation of International Application No. PCT/JP2023/000047, filed on Jan. 5, 2023, which claims priority to Japanese Patent Application 2022-034270, filed on Mar. 7, 2022, which is incorporated herein by reference.

### TECHNICAL FIELD

The present disclosure relates to a braided structure, a tube structure, a tube structure for a catheter, and a method for manufacturing a braided structure.

### BACKGROUND

Braided structures such as those described in JP 2019-181271 A is known.

In the structure described in JP 2019-181271 A, a braid is formed in such a manner that fiber yarns cross each other at equal intervals over an entire length in an axial direction.

### SUMMARY

An object of the present disclosure is to provide a braided structure and the like having flexibility that differs depending on the area in an axial direction.

In one aspect according to the present disclosure, a braided structure includes a shaft, a first wire wound in a spiral shape around an axis of the shaft, and a second wire wound around the shaft in a direction opposite to that of the first wire in a first portion and wound around the shaft in a direction identical to that of the first wire in a second portion.

### BRIEF DESCRIPTION OF DRAWINGS

FIG. 1 is a schematic view of a catheter.

FIG. 2 is a cross-sectional view of a tube.

FIG. 3 is a schematic view of a wire layer.

FIG. 4 is a cross-sectional view of a cross section D-D in FIG. 3.

FIG. 5 is a schematic view for describing a method for manufacturing a catheter.

FIG. 6 is a schematic view for describing a method for manufacturing a catheter.

FIG. 7 is a flowchart illustrating a series of processes in a manufacturing method.

FIG. 8 is a schematic view of a catheter according to a modified example.

FIG. 9 is a schematic view of a catheter according to another modified example.

### DESCRIPTION OF EMBODIMENTS

A braided structure according to an embodiment of the present disclosure will be described below. The braided structure has a structure obtained by seamlessly connecting a first braided portion formed by crossing a plurality of wires and a second braided portion having a coil shape formed by orienting the plurality of wires in the same direction. By seamlessly connecting a plurality of types of braided portions obtained by different braiding methods, it is possible to adjust flexibility depending on the area, and reduce bending at connection portions between the braided portions. The braided structure according to the embodiment is incorporated into part of a catheter. Catheters include sheaths used to guide other medical devices (another catheter, balloon, or the like) into a treatment region inside a body (internal treatment region such as a digestive organ, a biliary duct, a blood vessel, or the like), or electrode catheters provided with electrodes at a distal end.

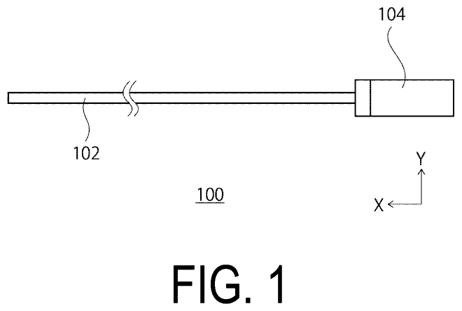

FIG. 1 is a schematic view of a catheter. As illustrated in FIG. 1, a catheter 100 includes a tube 102 which is long and narrow, and a base portion 104. Here, an X direction illustrated in FIG. 1 indicates an extending direction of the tube 102 in a state in which the tube 102 is straightened. The X direction is also referred to as an axial direction of the tube 102. Further, a Y direction indicates a direction orthogonal to the X direction (direction orthogonal in the paper plane).

The catheter 100 may be a deflectable (or steerable) catheter in which an area near a distal end of the tube 102 is operable by a user (surgeon), or a fixed catheter in which a shape of the tube 102 is fixed (that is, not operable by the user).

The base portion 104 functions as an operation portion for the user to operate the catheter 100. In this case, the base portion 104 is provided with a handle (not illustrated) for bending the area near the distal end of the tube 102 in a desired direction (in one direction or two directions). The user pushes or pulls the tube 102 into or out of the body by holding the base portion 104 and moving the base portion 104. At this time, the user can bend the area near the distal end of the tube 102 by operating the handle to adapt the shape of the tube 102 to a shape of a duct.

The base portion 104 may also function as a connector between the catheter 100 and other medical devices. When the base portion 104 functions as a connector, an accessory device such as a female lure or a hemostatic valve is connected to a rear end of the base portion 104. When the base portion 104 functions as a connector, a guide wire, an additional catheter, or the like may be inserted into the base portion 104.

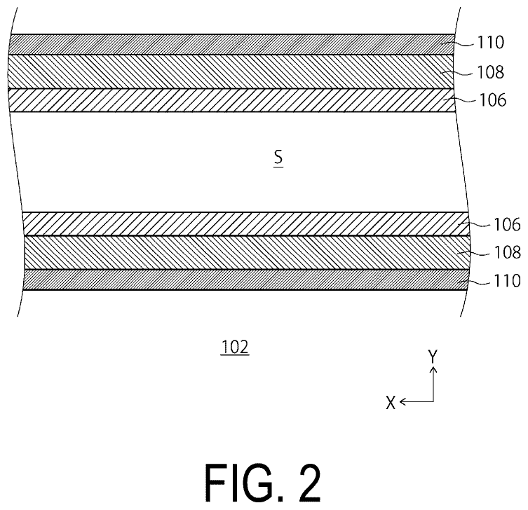

FIG. 2 is a cross-sectional view of the tube. Specifically, FIG. 2 is a cross-sectional view in an XY plane at a position along an axis of the tube. As illustrated in FIG. 2, a wall surface of the tube 102 has a three-layer structure. The tube 102 includes a shaft 106, a wire layer 108, and an outer layer 110. An internal space S of the tube 102 functions as a lumen for passing other medical devices, including a guide wire, therethrough.

The shaft 106 is formed from a tube body having flexibility. The shaft 106 is primarily used to help maintain a tubular shape of the wire layer 108 and/or to define the internal space S. The shaft 106 is formed from a resin material (for example, a thermoplastic elastomer such as PEBAX (trade name)) having a cylindrical shape. An inner circumferential surface of the shaft 106 (that is, surface defining the internal space S) preferably has a certain degree of slidability (that is, a low friction coefficient).

The wire layer 108 is a layer formed by wires, and is formed by winding at least two wires around an outer circumference of the shaft 106. The wire layer 108 being formed by a plurality of wires is not a dense layer formed from one material, such as a resin layer, for example, but a layer having spaces between the wires. The wire layer 108 can also be said to have a structure obtained by forming a braid of braided wires into a tubular shape or a cylindrical shape. The wire layer 108 can also be referred to as a braided layer formed by braiding wires. A detailed configuration of the wire layer 108 will be described below.

The outer layer 110 protects the wire layer 108. The outer layer 110 is formed from a biocompatible material such as an elastomeric resin. In the illustrated example, the outer layer 110 has a tubular structure covering an outer surface of the wire layer 108. The outer layer 110 may be formed integrally with the wire layer 108. That is, the wire layer 108 may be formed in the outer layer 110 to form an integrated body. A distal end tip that prevents tissue damage when the distal end of the tube 102 comes into contact with the tissue may be formed integrally with the outer layer 110.

The wire may be formed from a biocompatible metal (tungsten, stainless steel, or nickel titanium, for example) or an alloy, or other biocompatible material (nylon, polyether ketone resin (PEEK), liquid crystal polymer (LCP) resin). The wire may be formed by coating a predetermined material with a biocompatible material. The material of the wire can be selected as appropriate in accordance with the application of the braided structure. The plurality of wires may be made of different materials. The wire may have a circular cross section or may have a rectangular cross section.

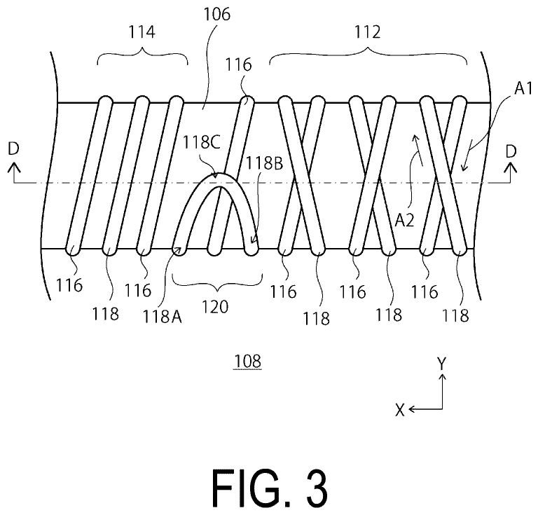

FIG. 3 is a schematic view of the wire layer. Specifically, FIG. 3 illustrates a schematic top view (view from the Y direction) of the wire layer. Note that, in the following description, it is assumed that the wire layer is constituted by two wires in order to simplify the description. The wire layer need only be constituted by at least two wires, and the number of wires is not limited to two. That is, a plurality of “first wires” or a plurality of “second wires” may be provided, and the number of “first wires” and the number of “second wires” may be different from each other.

As illustrated in FIG. 3, the wire layer 108 includes a first portion 112 and a second portion 114 in the axial direction. The first portion 112 and the second portion 114 differ in the way that the two wires (hereinafter, also referred to as “first wire 116” and “second wire 118”) are wound. Accordingly, a flexibility (at least one of a bending elastic modulus [MPa] or a minimum bending radius [mm]) of the tube 102 (refer to FIG. 1) in the first portion 112 differs from a flexibility of the tube 102 in the second portion 114. Further, the first portion 112 and the second portion 114 differ in torqueability (torsional strength around the axis: [N·m]). The first wire 116 and the second wire 118 are oriented in different directions in the first portion 112, making the first portion 112 have higher torqueability than the second portion 114. On the other hand, the first wire 116 and the second wire 118 are oriented in the same direction in the second portion 114, making the second portion 114 more readily bend (that is, have higher flexibility) than the first portion 112.

The first portion 112 and the second portion 114 are regions adjacent to each other in the axial direction, and both portions are seamlessly connected to each other. “Seamlessly connected” means that the first portion 112 and the second portion 114 are constituted by the same wire. Accordingly, no ends of the wire are present between the first portion 112 and the second portion 114. An arrangement of the first portion 112 and the second portion 114 is not particularly limited, and the first portion 112 may be closer to the distal end side or the second portion 114 may be closer to the distal end side. A plurality of the first portions 112 and a plurality of the second portions 114 may be provided. The arrangement of the first portion 112 and the second portion 114 in the axial direction can be selected as appropriate to provide physical properties (flexibility and/or torqueability) suitable for the application of the catheter 100.

In the first portion 112, the first wire 116 and the second wire 118 are wound around the shaft 106 so as to form a braided structure in which the first wire 116 and the second wire 118 cross each other. An overlapping order (that is, arrangement in a radial direction) of the first wire 116 and the second wire 118 can be selected as appropriate, and may be the same at all crossing points or may be different at each crossing point. The overlapping order being different at each crossing point means that the order is patterned such that the first wire 116 is positioned outward in the radial direction at a certain crossing point, the second wire 118 is positioned outward in the radial direction at an adjacent crossing point, and the first wire 116 is positioned outward in the radial direction at a further adjacent crossing point. The first wire 116 is wound in a spiral shape around the shaft 106 in a first direction A1 (clockwise direction around an axis of the shaft 106). In general, the term “spiral shape” refers to a shape that moves in a direction having a vertical component (component along the X axis) with respect to a rotation plane (circular cross section of the shaft 106). In the present disclosure, when reference is made to the “spiral shape,” an amount of movement in a certain direction having the vertical component need not be constant.

In the first portion 112, the second wire 118 is wound in a spiral shape around the shaft 106 in a second direction A2 (clockwise direction around the axis of the shaft 106) opposite to that of the first wire 116. As long as the first wire 116 and the second wire 118 have the same winding interval (amount of components along the X axis), the first wire 116 and the second wire 118 cross each other at equal intervals in the first portion 112.

In the second portion 114, the first wire 116 and the second wire 118 are wound in a spiral shape in the same direction (first direction A1) with respect to the axis of the shaft 106. That is, in the second portion 114, the first wire 116 and the second wire 118 are wound around the shaft 106, forming a braided structure in which the first wire 116 and the second wire 118 are substantially parallel to each other. The first wire 116 is wound in the same direction (first direction A1) from the first portion 112 to the second portion 114. The second wire 118 is wound in the second portion 114 in a direction opposite to that of the first portion 112. In the second portion 114, the first wire 116 and the second wire 118 are alternately arranged along an axis of the catheter 100 without crossing each other. The first wire 116 and the second wire 118 extend substantially parallel to each other. The phrase “substantially parallel” as used herein means that the first wire 116 and the second wire 118 do not cross each other, and does not necessarily mean that the distance between the two wires is constant. The second wire 118 can also be said to be wound in the opposite direction between the first portion 112 and the second portion 114.

The winding interval of the first wire 116 and/or the second wire 118 may differ between the first portion 112 and the second portion 114. Thus, the physical properties can be adjusted between the first portion 112 and the second portion 114.

A changing portion 120 is provided between the first portion 112 and the second portion 114. The changing portion 120 seamlessly connects the first portion 112 and the second portion 114. The phrase “seamlessly connects” means connecting so that no end of the first wire 116 or the second wire 118 is present. “no end is present” means that ends of the first wire 116 and ends of the second wire 118 are not present in that section and, for example, in a case in which the first wire 116 is formed by welding the ends of two wires, the welded portion is not present in that section. When the first wire 116 and the second wire 118 are seamlessly connected, the catheter 100 is less likely to break between the first portion 112 and the second portion 114.

In a case in which the winding interval of the first wire 116 differs between the first portion 112 and the second portion 114, preferably the winding interval of the first wire 116 gradually changes in the changing portion 120. This makes it possible to prevent the physical properties from abruptly changing in the changing portion 120. The winding interval of the first wire 116 may be narrowed in the changing portion 120 (may be made narrower than the winding interval in the first portion 112 and the second portion 114). This make it possible to reduce the bendability of the changing portion 120 and stiffen the changing portion 120 (that is, increase the bending elastic modulus).

In the changing portion 120, the winding direction of the second wire 118 changes. The winding direction of the second wire 118 is reversed in the changing portion 120. The second wire 118 includes a first wire portion 118A extending in the first direction A1, a second wire portion 118B extending in the second direction, and a third wire portion 118C between the first wire portion 118A and the second wire portion 118B. The third wire portion 118C has a curved shape (substantially U-shape). Microscopically, the third wire portion 118C includes components parallel to the X axis. That is, the third wire portion 118C includes components having an inclination with respect to the X axis that is 0 or substantially 0. The third wire portion 118C facilitates transmission of a force in the axial direction from the first wire portion 118A to the second wire portion 118B.

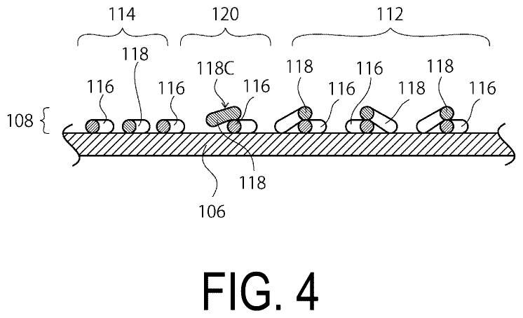

FIG. 4 is a cross-sectional view of a cross section D-D in FIG. 3. As illustrated in FIG. 4, a thickness of the wire layer 108 differs between the first portion 112 and the second portion 114. The thickness of the wire layer 108 as described herein is a parameter that depends on thicknesses of the first wire 116 and the second wire 118 (thicknesses in the radial direction of the shaft 106, and diameters of the wires when the wires have a circular cross section). In the first portion 112, there exist a portion in which the first wire 116 and the second wire 118 overlap each other (cross each other in a top view) and a portion in which the first wire 116 and the second wire 118 do not overlap each other. The thickness of the wire layer 108 in the first portion 112 refers to a portion having a minimum thickness (length in a Y-axis direction) within the overlapping portion of the first wire 116 and the second wire 118. That is, the thickness of the wire layer 108 in the first portion 112 is measured at a portion where the first wire 116, the second wire 118, and the shaft 106 are in close contact with each other. Accordingly, the thickness of the wire layer 108 in the first portion 112 corresponds to a sum of the thickness of the first wire 116 and the thickness of the second wire 118. For the thickness of the wire layer 108 in the first portion 112, even if the first wire 116 and the second wire 118 overlap, portions where a gap exists on an outer surface of the first wire 116, the second wire 118, or the shaft 106 should not be taken into account. In the second portion 114, only non-overlapping portions of the first wire 116 and the second wire 118 exist. Accordingly, the thickness of the wire layer 108 in the second portion 114 corresponds to the thickness of the first wire 116 or the second wire 118.

It is clear that the first portion 112 is thicker than the second portion 114. The first portion 112 being thicker than the second portion 114 means that the thickness at at least one location of the first portion 112 is thicker than the thickness at at least one location of the second portion 114 according to the definition described above. In a case in which the thicknesses of the first wire 116 and the second wire 118 are the same, the thickness of the first portion 112 is twice the thickness of the second portion 114.

With the thicknesses of the first portion 112 and the second portion 114 being different from each other, a diameter of the catheter 100 as a whole varies depending on the area. That is, a diameter of the first portion 112 is relatively large, and a diameter of the second portion 114 is relatively small. Accordingly, according to the embodiment, it can also be said that a diameter of the catheter 100 is adjusted by adjusting the winding directions of the first wire 116 and the second wire 118.

In the embodiment, the wire layer 108 is constituted by two wires, and thus a thickness of the changing portion 120 is also the same as that of the first portion 112. However, in a case in which the number of wires is increased, the wires may overlap one another depending on how the wires are bent, thereby increasing the thickness of the changing portion 120.

As described above, according to the embodiment, the flexibilities of the first portion 112 and the second portion 114 can be made different from each other.

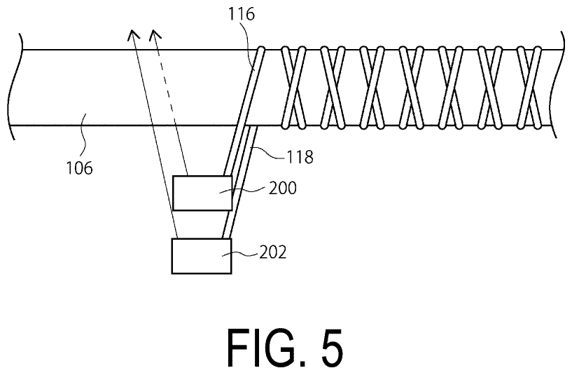

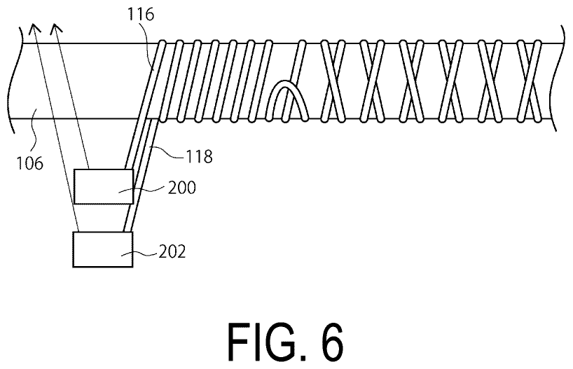

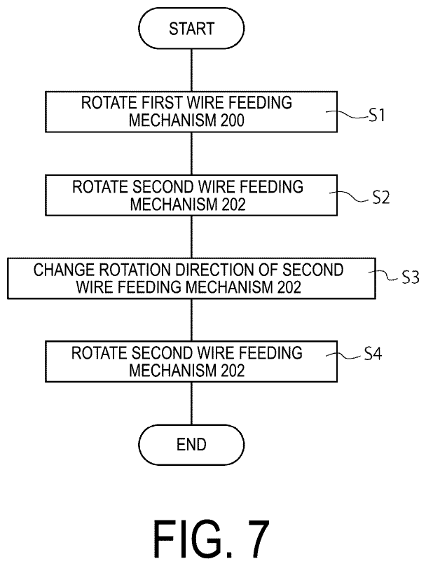

Hereinafter, a method for manufacturing a catheter as a braided structure will be described. FIG. 5 and FIG. 6 are schematic views for explaining the method for manufacturing a catheter. FIG. 7 is a flowchart illustrating a series of processes of the manufacturing method.

As illustrated in FIG. 5 and FIG. 6, to manufacture the catheter 100, two braiders (first wire feeding mechanism 200 and second wire feeding mechanism 202) are prepared. Each of the wire feeding mechanisms 200 and 202 independently rotates around the axis of the shaft 106. In a case in which the shaft 106 has a tubular structure, the shaft 106 may be fixed to a mandrel.

With reference to FIG. 5 to FIG. 7, the method for manufacturing the catheter 100 includes:
a step S1 of winding the first wire 116 in a spiral shape around the axis of the shaft 106 by using the first wire feeding mechanism 200,
a step S2 of winding the second wire 118 around the shaft 106 in a direction opposite to that of the first wire by using the second wire feeding mechanism 202,
a step S3 of changing a rotation direction of the second wire feeding mechanism 202 and changing a rotation direction of the spiral of the second wire 118, and
a step S4 of winding the second wire 118 around the shaft 106 in a direction identical to that of the first wire 116.

In step S1, the first wire feeding mechanism 200 is rotated around the axis of the shaft 106 to wind the first wire 116 around the shaft 106. The first wire feeding mechanism 200 rotates around the axis of the shaft 106 while moving in the axial direction of the shaft 106. Step S1 is continued until the series of processes is completed.

In step S2, the second wire feeding mechanism 202 is rotated around the axis of the shaft 106 to wind the second wire 118 around the shaft 106. The second wire feeding mechanism 202 rotates around the axis of the shaft 106 while moving in the axial direction of the shaft 106 with a delay from the first wire feeding mechanism 200. The rotation direction of the second wire feeding mechanism 202 is opposite to the rotation direction of the first wire feeding mechanism 200.

In step S3, the rotation direction of the second wire feeding mechanism 202 is changed. During this time, a moving speed of the second wire feeding mechanism 202 in the axial direction is the same as that in step S2. As a result, the third wire portion 118C that is curved (refer to FIG. 3) is formed in the second wire 118.

In step S4, the second wire feeding mechanism 202 is rotated around the axis of the shaft 106 to wind the second wire 118 around the shaft 106. The rotation direction of the second wire feeding mechanism 202 at this time is the direction after the rotation direction is changed in step S3. A delay amount of the second wire feeding mechanism 202 is maintained, making it possible to wind the first wire 116 and the second wire 118 in parallel. In a case in which the shaft 106 is molded on an outer periphery of a mandrel, the catheter 100 can be obtained by pulling out the mandrel from the shaft 106.

The disclosure is not limited to the embodiment described above, and each configuration of the embodiment may be modified as appropriate without departing from the spirit of the disclosure.

A material of the wire can be selected as appropriate in accordance with the application of the braided structure. For example, in a case in which the braided structure is used for an application requiring strength and flexibility (for example, a shaft of a golf club, a rod of a fishing rod, or the like), carbon fibers can be used as the wire. As the wire, various wires such as a wire made of metal and a wire made of resin may be used.

The shaft may be solid. In a case in which the shaft is solid, the shaft itself can function as a mandrel, eliminating the need to use a mandrel in the manufacturing process.

Modified examples such as the following are also within the scope of the present disclosure. A braided structure according to a modified example has a structure in which a first braided portion formed by crossing a plurality of wires and a second braided portion formed by orienting the plurality of wires in the same direction are seamlessly connected. Unlike the embodiment, in the modified example, the second braided portion is not formed by winding the wires in a coil shape, but by changing only the winding direction of a portion of the plurality of wires of the first braided portion.

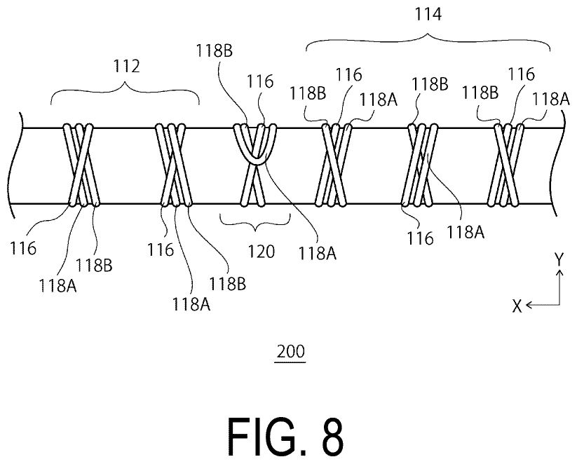

FIG. 8 is a schematic view of a catheter according to the modified example. As illustrated in FIG. 8, the catheter 200 is configured by seamlessly connecting the first portion 112 and the second portion 114 via a changing portion 120. The first portion 112 includes one first wire 116 wound in the first direction and two second wires 118A, 118B wound in the second direction. In the changing portion 120, the winding direction of only one of the second wires 118A, 118B (second wire 118A in the illustrated example) is changed. In the second portion 114, the first wire 116 and the second wire 118A are wound in the first direction, and the second wire 118B is wound in the second direction. That is, the first wire 116 is wound in the same direction from the first portion 112 to the second portion 114, and the winding direction of only a portion of the plurality of second wires (second wire 118A in the illustrated example) is changed in the changing portion 120.

With the braided structure being different between the first portion 112 and the second portion 114, the flexibility is different between the first portion 112 and the second portion 114. More specifically, the flexibility in a ±Y direction differs between the first portion 112 and the second portion 114. That is, in the first portion 112, the flexibility in the +Y direction is greater than the flexibility in the −Y direction. On the other hand, in the second portion 114, the flexibility in the −Y direction is greater than the flexibility in the +Y direction.

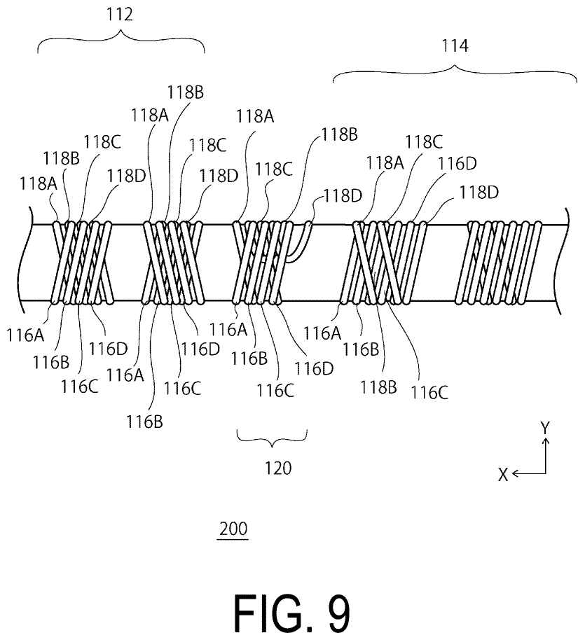

FIG. 9 is a schematic view of a catheter according to another modified example. As illustrated in FIG. 9, a catheter 300 is configured by seamlessly connecting the first portion 112 and the second portion 114 via the changing portion 120. The first portion 112 includes four first wires 116A to 116D wound in the first direction and four second wires 118A to 118D wound in the second direction. In the changing portion 120, the winding direction of only a portion of the second wires 118A to 118D (second wires 118B, 118D in the illustrated example) is changed. In the second portion 114, the first wires 116A to 116D and the second wires 118B and 118D are wound in the first direction, and the second wires 118A and 118C are wound in the second direction. That is, the first wire 116 is wound in the same direction from the first portion 112 to the second portion 114, and the winding direction of only a portion of the plurality of second wires (second wires 118B and 118D in the illustrated example) is changed in the changing portion 120. Note that the wires for which the winding direction is changed can be determined as appropriate. For example, the winding direction of the second wires 118A and 118D may be changed.

With the braided structure being different between the first portion 112 and the second portion 114, the flexibility is different between the first portion 112 and the second portion 114. More specifically, the flexibility in the ±Y direction differs between the first portion 112 and the second portion 114. That is, in the first portion 112, the flexibility in the +Y direction is greater than the flexibility in the −Y direction. On the other hand, in the second portion 114, the flexibility in the −Y direction is greater than the flexibility in the +Y direction.

The present disclosure can be utilized in the fields of a braided structure, a tube structure, a tube structure for a catheter, and a method for manufacturing a braided structure.

While preferred embodiments of the disclosure have been described above, it is to be understood that variations and modifications will be apparent to those skilled in the art without departing from the scope and spirit of the disclosure. The scope of the disclosure, therefore, is to be determined solely by the following claims.

1. A braided structure comprising:
a shaft;
a first wire wound in a spiral shape around an axis of the shaft; and
a second wire wound around the shaft in a direction opposite to that of the first wire in a first portion, and wound around the shaft in a direction identical to that of the first wire in a second portion.

2. The braided structure according to claim 1, wherein
the second wire is provided with a changing portion at which a rotation direction of a spiral changes.

3. The braided structure according to claim 1, wherein
the second wire does not have any ends present in at least the first portion and the second portion.

4. The braided structure according to claim 1, wherein
the first wire and the second wire form a wire layer around the shaft, and
a thickness of the wire layer in the first portion is thicker than a thickness of the wire layer in the second portion.

5. The braided structure according to claim 4, wherein
a difference between the thickness of the wire layer in the first portion and the thickness of the wire layer in the second portion is substantially equal to a thickness of the second wire.

6. The braided structure according to claim 1, wherein
a winding interval of the first wire differs between the first portion and the second portion.

7. A braided structure comprising:
a first portion in which a first wire and a second wire are wound around a shaft so as to cross each other;
a second portion in which the first wire and the second wire are wound around the shaft in an identical direction; and
a changing portion disposed between the first portion and the second portion and at which a direction of the second wire is reversed,
the first portion, the second portion, and the changing portion being provided along an axis of the shaft.

8. A method for manufacturing a braided structure, the method comprising:
winding a first wire in a spiral shape around an axis of a shaft;
winding a second wire around the shaft in a direction opposite to that of the first wire; and
winding the second wire around the shaft in a direction identical to that of the first wire.

9. The method for manufacturing a braided structure according to claim 8, further comprising:
changing a rotation direction of a spiral of the second wire after the winding of the second wire in the direction opposite to that of the first wire.
